(19) United States
(12) Patent Application Publication
(10) Pub. No.: US 2015/0105657 A1
(43) Pub. Date: Apr. 16, 2015

(54) Method and kit for treatment of dysphagia

(71) Applicant: Maggie Scott, Surfside Beach, TX (US)

(72) Inventor: Maggie Scott, Surfside Beach, TX (US)

(21) Appl. No.: 14/052,556

(22) Filed: Oct. 11, 2013

(51) Int. Cl. A61B 6/12

(52) U.S. Cl. CPC A61B 6/12
USPC 600/431; 600/593

(57) ABSTRACT

Described is a method and products for testing patients having difficulty swallowing specialized capsules. Capsules of various sizes are manufactured. One group of capsules, gels or tablets is manufactured containing s non-active composition and the second group of capsules, gels or tablets are manufactured using an X-Ray imaging agent. The method comprises testing the patient swallowing difficulty utilizing each group of products.

### BACKGROUND OF INVENTION

The present invention relates to a method of treating and evaluating people with swallowing problems. In particular, the present invention relates to a method of treating and evaluating patients having difficulty swallowing tablets and capsules.

Dysphagia is defined normally, as difficulty swallowing. With dysphagia, patients often feel a sensation that suggests a difficulty in the passage of solids or liquids form the mouth into the stomach.

Swallowing problems are generally found in elderly patients, people who have had strokes, or people with head and neck cancer or neurological diseases.

In the prior art there are methods and devices for treating dysphagia disclosed.

However, currently there is no method available for speech pathologists to specifically test and treat for people having problems swallowing their medications.

### SUMMARY OF THE INVENTION

Accordingly, the present invention provides a method and materials for testing and treating patients who may be having difficulty swallowing capsules or tablets. Capsules of various sizes are manufactured. One group of capsules are manufactured containing placebo and the second group of capsules are manufactured using barium. The method comprises testing the patient swallowing difficulty utilizing each group of capsules.

One major goal of the present invention is to measure the level of difficulty swallowing medications. The method is performed with radiographic imaging equipment and without radiographic equipment

Other features and advantages of the present invention will be apparent to those skilled in the art from reading the specifications in conjunction with the drawings.

### BRIEF DESCRIPTION OF THE DRAWINGS

In the drawings,

FIG. 1 is a sample of different sizes and weight of the medications.

FIG. 2 is a sample of capsules from 00,000, 0, 1, 2, 3, 4 and 5.

FIG. 3 is a sample of each product ranging in size.

FIG. 4 is a sample of capsules showing the diameter and volume in metric.

FIG. 5 is a sample of capsules showing the diameter and volume in inches.

### DETAILED DESCRIPTION OF PREFERRED EMBODIMENTS

A speech pathologist is normally the person first called to evaluate the patient having swallowing problems. This person obtains the medical history and performs a physical exam. A patient needing further examination then the patient will be tested with food of different consistencies and liquids. During the swallowing phase the pathologist makes detail accounting of any delay or deviations of the normal swallowing pattern. Then the patient may have a Modified Barium Swallow (MBS) study which involves providing food and liquid and utilizing X-ray to determine the level of swallow function.

The method of the present invention evaluates or tests the specific problem of swallowing medications which have a different consistency and viscosity then food. Food is normally broken down through the saliva in the mouth. However, medications are specifically coated to prevent the breakdown within the mouth. Thus, patients can have difficulty swallowing capsules or tablets and not food.

The present invention provides a method and product that allows a speech pathologist to evaluate, test and treat swallow function deficits with capsules, caplets and tablets. Based upon the evaluation utilizing the method and the product of the present invention, a speech pathologist can setup a specific medication plan to suit a particular patient.

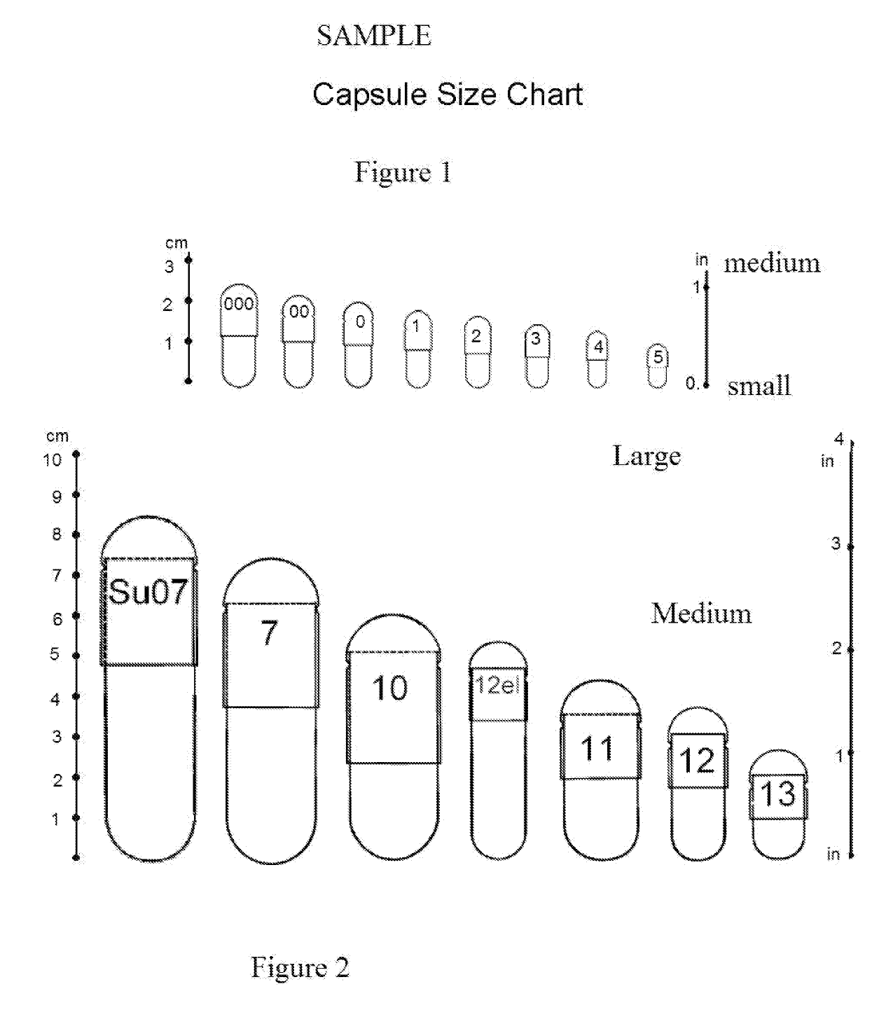

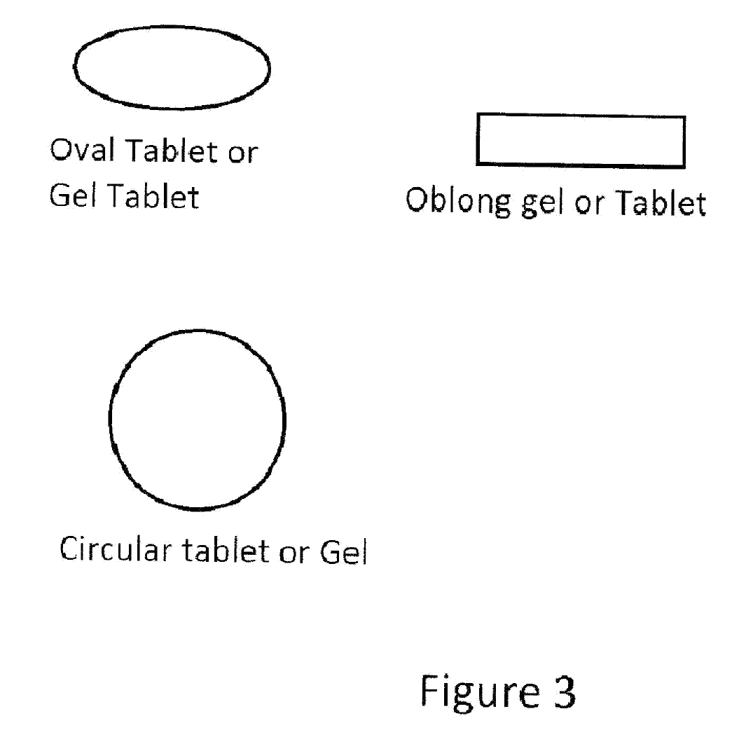

Products are broken down into the following types:
Hard shelled capsules (20) which is an encapsulation of a dry powder form or miniature pellets with or without barium as shown in FIGS. 1 and 2.
Soft shell capsules (25) which is an encapsulation in an oil form; soft shell capsules can be manufactured as shown in FIGS. 1 and 2; additionally soft shell capsules can take the form as shown in FIG. 3.
Tablets (10) is in a powdered pressed compacted solid form as shown in FIG. 3; and
Caplets (5) is a coated tablet in the shape of a capsule as shown in FIGS. 1 and 2; a smooth-coated tablet shaped like a capsule, used as a tamper-resistant alternative to a capsule, or an easy-to-swallow alternative to regular tablets.

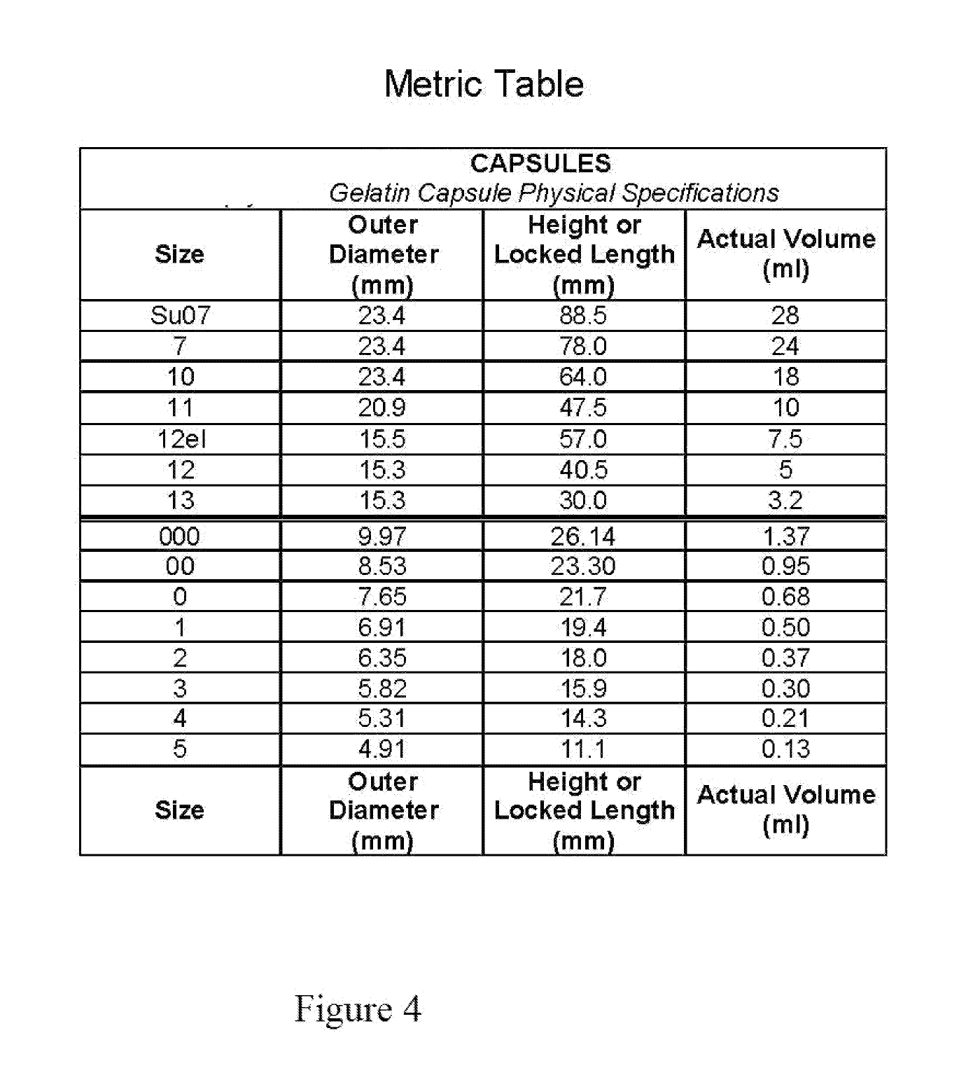

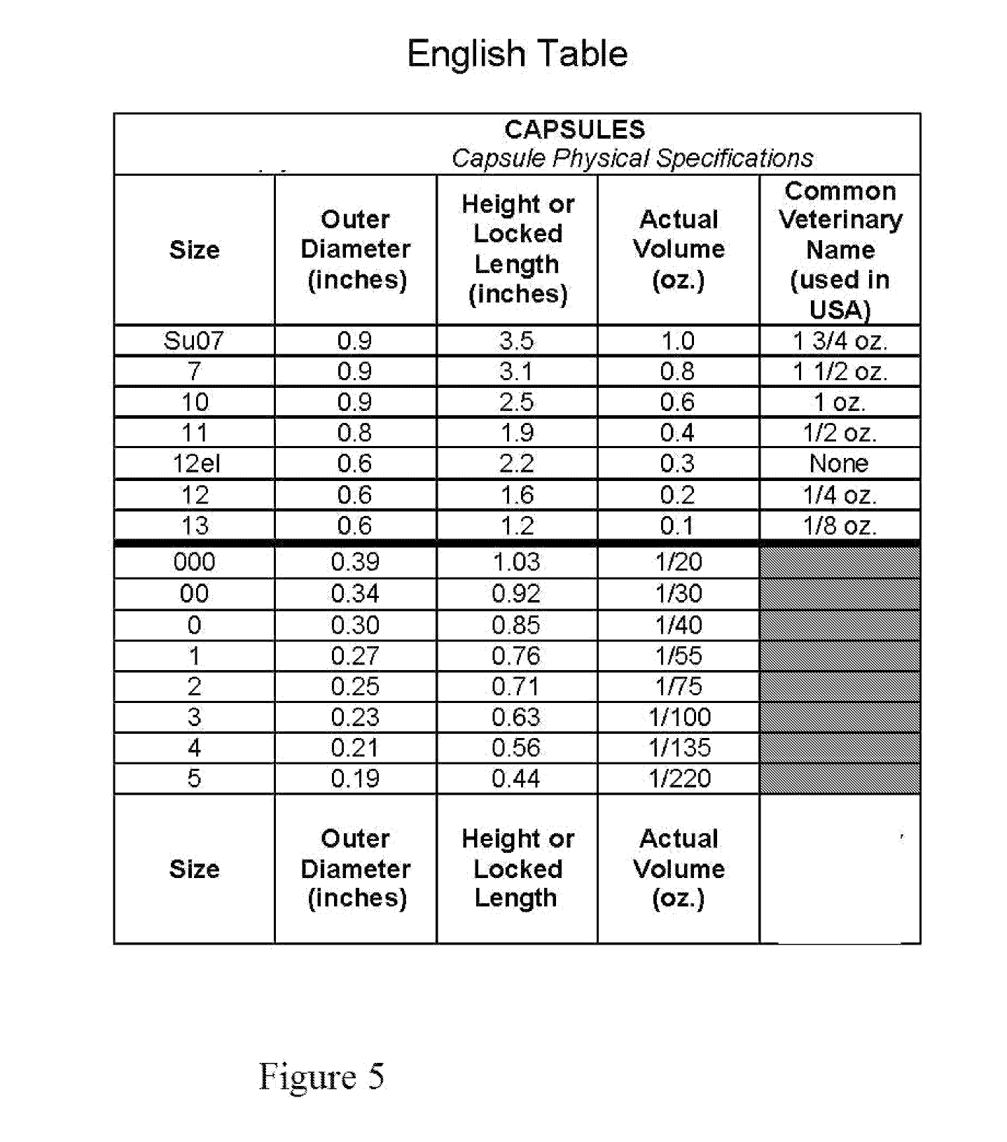

Tablets, caplets, capsules, and soft gels are manufactured in various sizes, shapes and volumes as shown in FIGS. 4 and 5. FIG. 1 shows the different sizes and weight for capsules. As shown in FIG. 2 there are 8 different sizes of capsules 00, 000, 0, 1, 2, 3, 4 and 5. As shown in FIG. 1, the density of the powder within the hard shell capsules various from 60 to 615 mg for light density, 90 to 960 mg for normal density and 130 to 1370 for heavy density, the volume of the hard shell capsules various from 0.13 to 1.37 ml and the locked length varies from 11.10 to 26.14 in length. Because caplet is formed in the shape of a hard shell capsule, it would vary in size, shape, and length as illustrated in FIGS. 1 and 2. The present invention can be single piece encapsulation or two piece gel encapsulation sealed in a gelatin solution. Any pill manufactured over 10 centimeters is considered extra large. As shown in FIG. 4 the capsules diameter can range from 4.91 to 23.4 millimeters with a volume ranging between 0.13 to 28 milliliters.

In the present invention the above mentioned products are manufactured in different sizes with and without barium sulfate from extra small, small, medium, large and extra large based upon the chart in FIGS. 1 and 2. The first group of products are manufactured without barium sulfate and the second group is manufactured with barium sulfate.

In the present invention—placebo gelatin substance is used to fill the;

Based upon the patient treatment guidelines, a group of capsules ranging from small to large is created based upon the charts shown in FIGS. 1, 2 and 3. The group can consist of hard shell or soft shell capsules, tablets, or caplets. The treatment consist of tracking the patient swallowing each type of pill in the group create. The pathologist tracks the Speech pathologist can also utilize the product during therapy sessions to train compensatory strategies for taking medications.

The first group of medications uses a non active composition that is created in a powdered form or in a gel form. The non-active compositions are created in powder form for tablets, hard shell capsules, caplets. Then, a non-active gel type composition is created for the soft gel type capsules. Various sizes of the non-active compositions mentioned above are manufactured for use when X-Ray is not utilized.

The second group of medications uses an imaging agent such as barium sulfate which is the radio contrast agent for X-ray imaging equipment. Barium Sulfate compositions are created in powder form for tablets, hard shell capsules, caplets. Then, a Barium Sulfate Composition in a gel type composition is created for the soft gel type capsules. Various sizes of the product containing barium sulfate are manufactured.

Normally, food cannot be used to test the level of difficulty a patient is having swallowing medications. There is nothing for the doctor to use to test whether or not medications needs to be crushed or provided in liquid form to the patients.

To test the level of difficulty of swallowing, the following steps are performed:
1. performing a bedside physical examination to determine the level of difficulty of a patient having difficulty swallowing using the following steps: (1) labials evaluation which includes evaluation of the tightening of the lips; (2) lingual function which includes tongue evaluation; and (3) laryngeal function which includes voice box evaluation; this testing includes dealing with the sensation of the medications being stuck in throat.
2. based upon the physical examination creating a kit of products consisting of a first group of products and a second group of products;
3. providing a first group of products with a non-active composition;
4. based upon the physical examination, creating at least one subset within the first group of products consisting of tablets, hard shell capsules, soft shell capsules or caplets from extra small to large;
5. providing the patient with a selected subset from the first group of products;
6. starting with the extra small size within the selected subset within the first group of products;
7. allowing the patient to swallow each product within the selected subset in the first group of the products;
8. recording the results of the level of difficulty of swallowing; and
9. repeating steps 5 to 9 for each subset within the first group of products.

To test the next level of difficulty of swallowing, the following steps are performed:
1. providing the patient with a second group of products having an imaging agent such as barium;
2. based upon the physical examination, at least one subset within the second group of products consisting of tablets, hard shell capsules, soft shell capsules or caplets from extra small to large;
3. providing the patient with a selected subset within the second group of products;
4. starting with the extra small size within the at least one subset within the second group of products;
5. allowing the patient to swallow each product within selected subset within the second group of the products;
6. x-raying the patient during the swallowing process of each product within the selected subset to detect the difficulty;
7. recording the results of the level of difficulty of swallowing; and
8. repeating steps 3 to 7 for each subset within the second group of products.

What is claimed is:

1. A kit for a speech pathologist to test or evaluate a patient having swallowing deficits of medicine, the kit including a plurality compositions of medications selected from the group consisting of
(a) a hard shell capsule containing a powdered substance of a non-active composition manufactured in various sizes;
(b) a soft shell capsule containing a gel substance of a non-active composition manufactured in various sizes;
(c) a tablet containing a compressed powdered substance of a non-active composition manufactured in various sizes;
(d) a non-active caplet containing a coated compressed powdered substance of a non-active composition manufactured in various sizes.

2. The kit of claim 1 wherein the plurality of medications contains a subset of the plurality of medications based upon a physical examination of the patient having swallowing difficulty of the medications.

3. The kit of claim 1 wherein the plurality of products are manufactured based upon a standard physical examination of the patient having swallowing difficulty.

4. The kit according to claim 1 wherein the non-active composition products include an imaging agent.

5. The kit according to claim 4 wherein the imaging agent is a barium sulfate-containing compound.

6. The kit according to claim 1 wherein the hard shell capsule is manufactured from a predetermined extra small size to a predetermined large size.

7. The kit according to claim 1 wherein the soft shell capsule is manufactured from a predetermined extra small size to a predetermined large size.

8. The kit according to claim 1 wherein the tablet is manufactured from a predetermined extra small size to a predetermined large size.

9. The kit according to claim 1 wherein the caplet is manufactured from a predetermined extra small size to a predetermined large size.

10. The kit according to claim 1 wherein the plurality of medications ranges in size between 0.1 to 1 Centimeter.

11. The kit according to claim 1 wherein the plurality of medications ranges in size between 1 to 3 Centimeter.

12. The kit according to claim 1 wherein the the plurality of medications ranges in size between 3 to 10 Centimeter.

13. The kit according to claim 1 wherein the plurality of medications ranges in outer diameter between 4.91 to 23.4 millimeters.

14. The kit according to claim 1 wherein the plurality of medications ranges in outer diameter between 0.13 to 28 milliliters.

15. A method for a speech pathologist to evaluate a patient having swallowing deficits of medications, the method comprising:
(1) providing medications from claim 1 selected from the first group of medications consisting of:
(a) a hard shell capsule containing a non-active composition of a powdered substance manufactured in various sizes;
(b) a soft shell capsule containing a non-active composition of a gel substance manufactured in various sizes;
(c) a tablet containing a non-active composition of a compressed powdered substance manufactured in various sizes;
(d) a non-active caplet containing a non-active composition of a coated compressed powdered substance manufactured in various sizes.
(2) based upon a physical examination by the speech pathologist, creating a plurality of subsets of medications ranging in size from an extra small predetermined size to an extra large predetermine size within the first group of products;
(3) providing the patient with a selected subset within the first group of medications;
(4) starting with the extra small size within the selected subset, allowing the patient to swallow each product in the selected subset within the first group of the medications;
(5) as the patient swallows, identifying the level of swallowing difficulty;
(6) recording the results of the level of difficulty of swallowing;
(7) progressively moving up in product size within the selected subset; and
(8) repeating steps (3) through (7) for each subset in the plurality of subsets of medications.

16. The method of claim 10 wherein the first group of medications contain an imaging agent.

17. The method of claim 11 wherein the imaging agent is Barium Sulfate.
